(19) United States
(12) Patent Application Publication
(10) Pub. No.: US 2012/0215158 A1
(43) Pub. Date: Aug. 23, 2012

(54) ENDOSCOPIC CAPS FOR IONIZED PLASMA CONFINEMENT, SHAPING AND CONTROL OF THERAPEUTIC PURPOSES

(75) Inventor: James S. Barthel, Tampa, FL (US)

(73) Assignee: H. LEE MOFFITT CANCER CENTER & RESEARCH INSTITUTE, INC., TAMPA, FL (US)

(21) Appl. No.: 13/390,895

(22) PCT Filed: Aug. 20, 2010
(86) PCT No.: PCT/US2010/002296

§ 371 Date: May 8, 2012

Related U.S. Application Data
(60) Provisional application No. 61/235,536, filed on Aug. 20, 2009.
(60) Provisional application No. 61/242,577, filed on Sep. 15, 2009.

(51) Int. Cl. A61M 37/00

(52) U.S. Cl. 604/26

(57) ABSTRACT

The subject invention concerns caps that fit on the end of an endoscope. Endoscopic caps of the invention can provide for the control and shaping of an ionized inert gas plasma for purposes of efficiently and precisely burning and removing tissue layers. The device can be used in the treatment of premalignant and malignant conditions, such as Barrett's esophagus and early esophageal cancer, as well as other therapeutic applications.

### CROSS-REFERENCE TO RELATED APPLICATIONS

The present application claims the benefit of U.S. Provisional Application Ser. No. 61/242,577, filed Sep. 15, 2009, and U.S. Provisional Application Ser. No. 61/235,536, filed Aug. 20, 2009, each of which is hereby incorporated by reference herein in its entirety, including any figures, tables, and drawings.

### FIELD OF INVENTION

This invention relates to a cap that fits on the end of a flexible or rigid endoscope that can be used to contain, control and shape ionized plasma and remove tissue coagulum for purposes of efficiently and precisely treating diseases and abnormalities that involve tissue layers.

### BACKGROUND OF THE INVENTION

Argon plasma coagulation (APC) is a monopolar non-contact electrosurgical method that transfers electrical energy to tissue by means of ionized non-thermal, inert gas plasma (Ginsberg et al. (2002)). Resistance in an electrical conductor produces heat. As tissue is heated by current flow, its electrical resistance increases. Electrical current flowing through the argon plasma and into tissue seeks the path of least resistance in accordance with the laws of electrophysics. This permits a superficial tissue injury effect that is free of mechanical contact artifacts and that is primarily a function of the shape of the ionized plasma, the stability of the distance over which the plasma must conduct current from its ignition source to the target tissue, and the homogeneity of the target tissue conductivity. Examples of endoscopic and argon plasma devices are described in U.S. Pat. Nos. 7,517,347; 6,210,410; and 6,063,084; and in published U.S. patent applications 2009/0024122 and 2007/0034211. Refinement of argon plasma tissue treatment methodology will improve the uniformity and superficiality of tissue layer thermal treatments and permit utilization of the therapeutic effects of active charged and uncharged molecules produced by the plasma.

### BRIEF SUMMARY OF THE INVENTION

The subject invention concerns a series, set, or collection of caps which can be fitted onto the distal end of a flexible or rigid endoscope. In one embodiment, as shown in FIGS. 1A and 1B, a cap of the invention comprises a generally cylindrical cap body having a distal end portion with a distal end opening and having a proximal end portion with a proximal end opening which can operably connect with the distal end of an endoscope. The wall of the cap body can optionally comprise one or more venting holes in the distal end portion of the cap body. A cap of the invention can optionally comprise a conduit on the exterior or the interior of the cap body for containing, for example, fluid, gas and/or ignition device wiring. In one embodiment, the wall of the distal end portion of the cap body is hollow, such that the distal end portion comprises an inner wall and an outer wall defining an internal hollow space in the cap body. In a specific embodiment, the conduit is directly and operably connected to the internal hollow space in the cap body. In a further embodiment, one or more fluid or gas delivery ports can be provided on the inner wall of the distal end portion having the internal hollow space.

The subject invention provides methods of ionized plasma tissue layer treatment utilizing a generally cylindrical cap of the invention fitted on the end of a flexible or rigid endoscope to facilitate low power non-thermal plasma ignition and maintenance, plasma confinement, and plasma behavior control. The invention encompasses specific modifications in cap shape, diameter, length, open end bevel or edge profile, gas delivery, venting, and plasma ignition device positioning for purposes of minimizing plasma ignition and maintenance power requirements, facilitating control of plasma behavior, and debridement of tissue coagulum, which are all important aspects of the new method.

### BRIEF DESCRIPTION OF THE DRAWINGS

FIGS. 1A-1C illustrate various embodiments of a cap of the invention having an angled cap wall and external conduit for routing of gas and ignition device wiring.

FIGS. 2A and 2B illustrate various embodiments of a cap of the invention with shouldered cap wall.

FIG. 3 illustrates an embodiment of a cap of the invention with spherical cap wall.

FIGS. 4A-4C illustrate various embodiments of a cap of the invention with beveled open cap end.

FIG. 5 illustrates an embodiment of a cap of the invention with hollow distal cap end wall for multiple fluid or gas delivery ports.

FIGS. 6A and 6B illustrate various embodiments of a cap of the invention with attached electromagnetic devices to produce osculation of the ferromagnetic ignition device support or direct shaping of plasma.

FIG. 7 illustrates an external conduit.

FIGS. 8A and 8B illustrate various embodiments of a cap of the invention with probe based gas delivery and ignition systems.

FIGS. 9A-9E illustrate distal end cap edge profiles that can enhance tissue debridement.

### DETAILED DESCRIPTION OF THE INVENTION

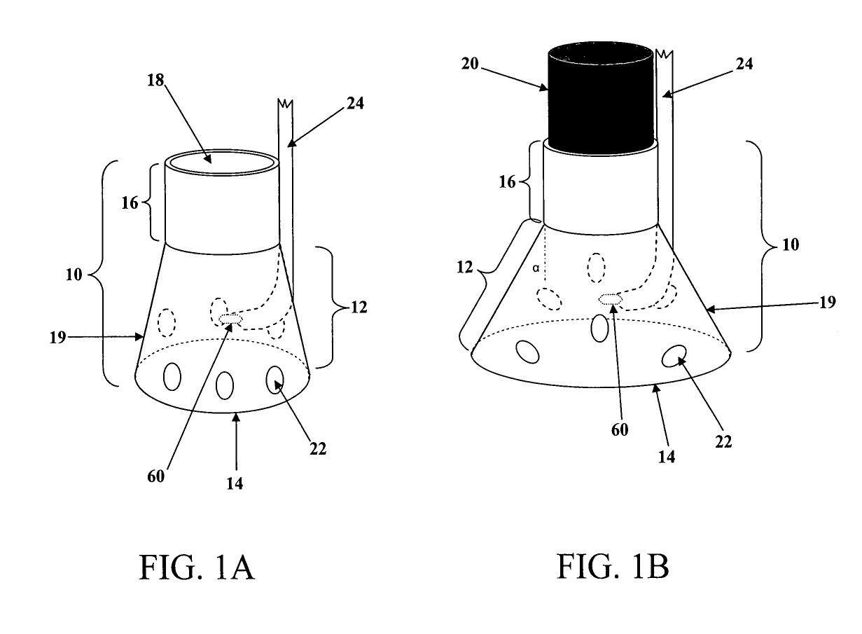

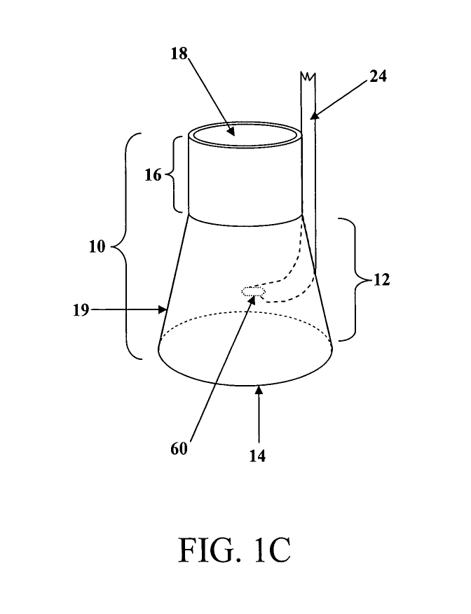

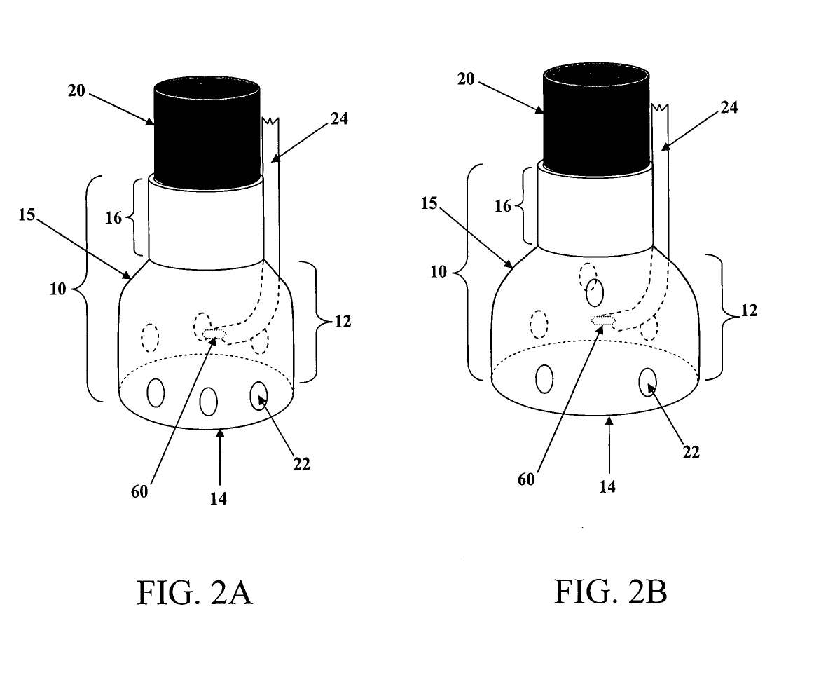

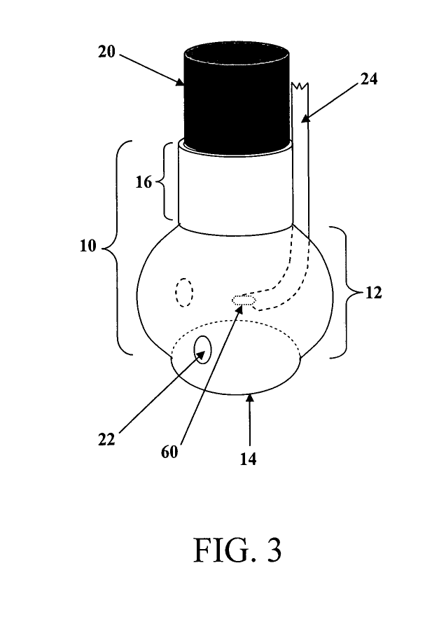

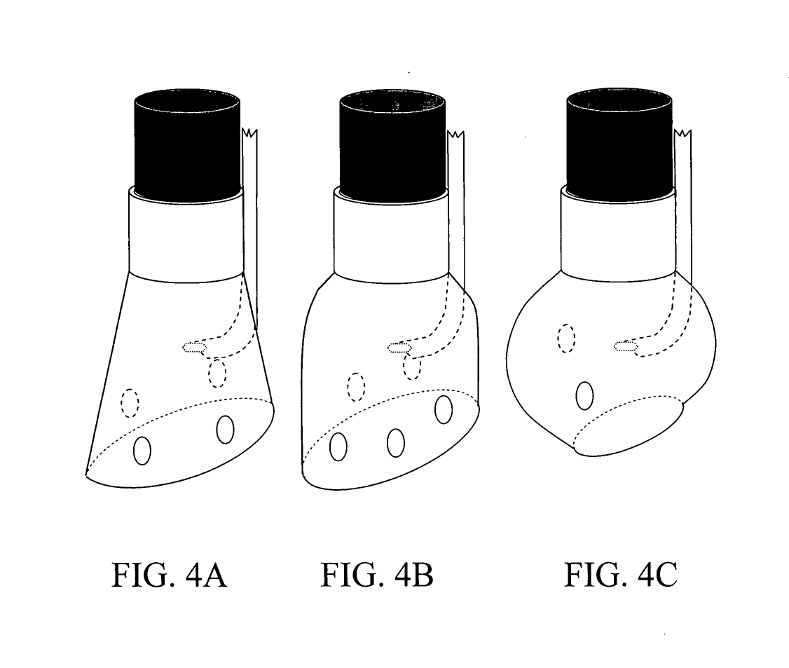

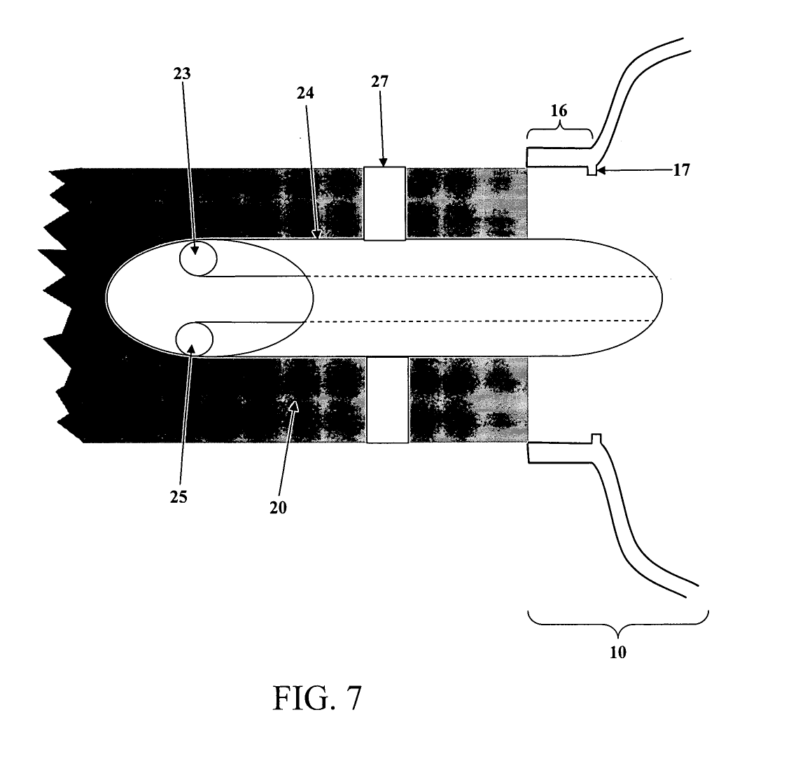

The subject invention concerns a series, set, or collection of caps which can be fitted onto the distal end of a flexible or rigid endoscope. In one embodiment, as shown in FIGS. 1A and 1B, a cap of the invention comprises a generally cylindrical and/or conical shaped cap body 10 having a distal end portion 12 with a distal end opening 14 and having a proximal end portion 16 with a proximal end opening 18 which can operably connect with the end of an endoscope 20. An annular rib 17 can optionally be provided on the interior of the proximal end portion 16 (see, for example, FIG. 7). The annular rib 17 can act as a stop for preventing the endoscope from being inserted beyond a certain point in the proximal end portion 16 of the cap body 10. The distal end portion 12 and the proximal end portion 16 are generally provided in a coaxial orientation. Typically, the diameter of the distal end opening 14 is larger than the diameter of the proximal end opening 18. In one embodiment, the cap body 10 is conical shaped. In a specific embodiment, the wall of the cap body 10 tapers from the proximal end to the distal end such that the distal end portion 12 of the cap body 10 is conical shaped, as shown in FIGS. 1A-1C. In another embodiment, the distal end portion 12 of the cap body 10 has a generally U-shaped appearance, having a generally angular or curved shoulder 15, as shown in FIGS. 2A and 2B. In another embodiment, the distal end portion 12 of the cap body 10 has a generally globe, spherical, or round shape, as shown in FIG. 3. The distal end opening 14 of cap body 10 can be of any desired angle, including from 0 to more than 45 degrees (see, for example, FIGS. 4A-4C) such that the plane of the distal end opening 14 is oblique to the insertion direction of the endoscope. The wall 19 of the cap body 10 can optionally comprise one or more venting holes 22 in the distal end portion 12 of the cap body 10. In another embodiment, the wall 19 of the cap body 10 does not comprise any venting holes (see, for example, FIG. 1C) regardless of the shape or construction of the distal end portion 12 of the cap body 10.

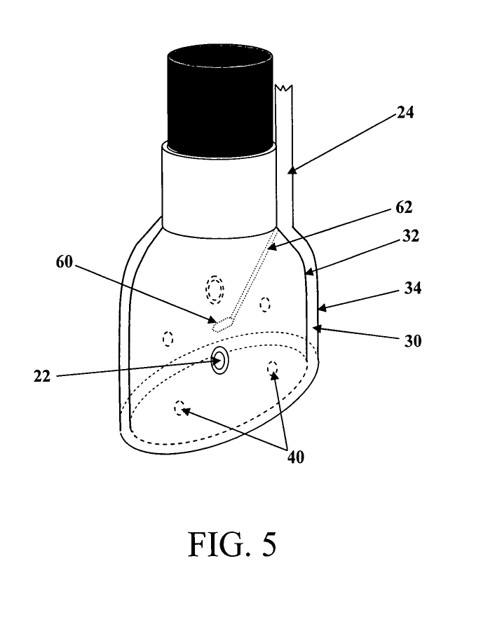

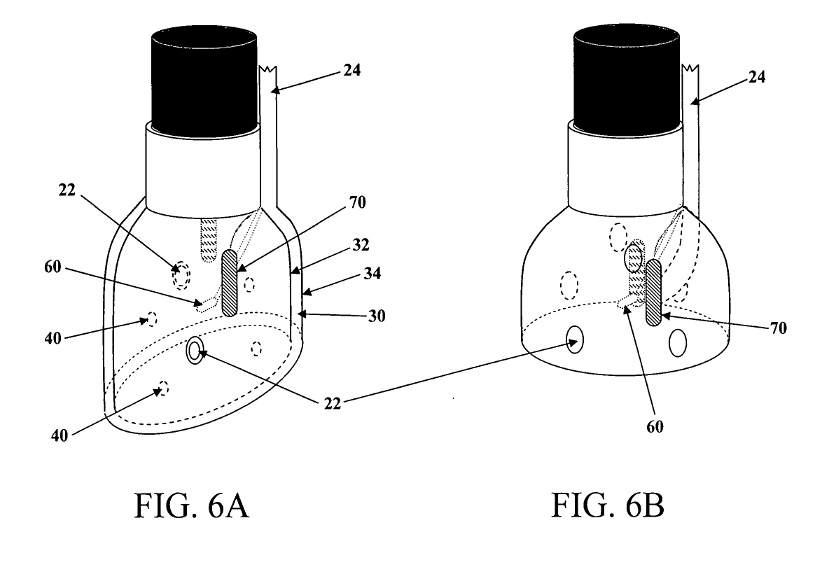

A cap of the invention can also optionally comprise a conduit 24 on the exterior and/or the interior of the cap body 10 for containing, for example, fluid, gas and/or ignition device wiring 23, and/or a wash tube for cleaning an ignition device 25, etc. The conduit 24 can extend in an orientation substantially axially along the exterior of the cap body 10. The conduit 24 can be releasably attached (via e.g., a strap 27) to the endoscope 20 or to cap body 10, or it can be more permanently attached, for example, by way of bonding, adhesive, or welding. The conduit 24 can be composed of a rigid or flexible material. In one embodiment, the wall of the distal end portion 12 of the cap body 10 is hollow, such that the distal end portion 12 comprises an inner wall 32 and an outer wall 34 defining an internal hollow space 30 in the cap body, as illustrated in FIG. 5 and FIG. 6A. In a specific embodiment, the conduit 24 is directly and operably connected to the internal hollow space 30.

A cap of the invention can also optionally comprise a releasably locking or securing means in the cap body 10 for locking or securing the cap in place when attached to the endoscope.

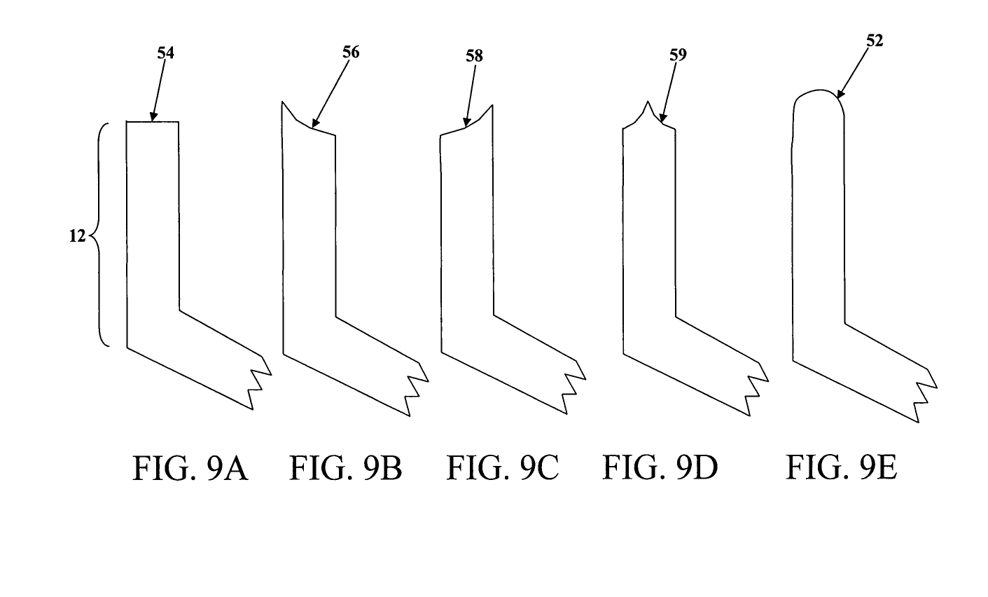

As illustrated in FIGS. 9A-9E, the cap of the invention can have a rounded edge 52, a square or flat edge 54, or an internal tapered edge 56, or an external tapered edge 58, or an internal and external tapered edge 59 on the distal end portion 12 of the cap body 10.

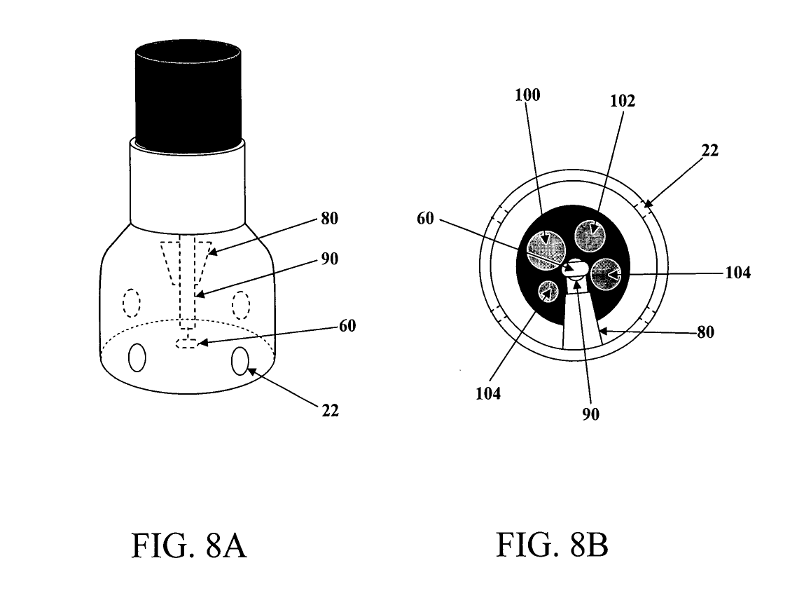

Caps of the invention can also comprise a plasma ignition device 60 provided inside of the distal portion of the cap body for producing an ionized plasma. Any suitable plasma ignition device in the art can be utilized with the subject invention. In one embodiment, a plasma ignition device is provided on a support 62. The support 62 can comprise, for example, an insulated or uninsulated ferromagnetic metal current conductor. Caps of the present invention can also include an electromagnetic device 70 attached to or embedded in the wall of the distal portion of the cap body 10 or positioned in sufficient proximity to the cap body 10 so as to provide an operator of the endoscope the ability to manipulate or adjust the shape of a cap confined plasma through the use of electromagnetic fields generated by the device (see, for example, FIGS. 6A and 6B). Any suitable electromagnetic device capable of generating an electromagnetic field for control of cap confined plasma can be utilized with the subject invention. In a further embodiment, one or more gas or fluid delivery ports 40 can be provided on the inner wall 32 of the distal end portion 12 having the internal hollow space 30, wherein the conduit 24 can be indirectly or directly and operably connected to the hollow space 30 and in communication therewith. Caps of the invention can also include a probe deflection shelf 80 attached to an inner wall 32 of the distal portion of the cap body 10, wherein the probe deflection shelf 80 can position a tip of a probe 90 toward the center of a field defined by the boundaries of the cap body. As shown in FIG. 8B, an endoscope for use with a cap of the invention can optionally comprise an objective lens 100, an air and/or water jet 102, and one or more lightguides 104.

A cap of the invention can be constructed of any suitable material, including for example, plastic, glass, metal or metal alloy, ceramic, etc. In one embodiment, a cap of the invention is constructed from a clear plastic material such as (but not limited to) polyethylene, polycarbonate, and polyurethane. The material selected for cap construction will depend on the degree of desired distal end stiffness and other variables associated with a particular component configuration. In a specific embodiment, a cap of the invention is made of a clear or translucent plastic material.

Endoscopic caps of the invention are contemplated to include all variations in cap diameters, length and shape, cap open end bevels and edge profiles, cap associated gas delivery and venting mechanisms, and plasma ignition device positions and configurations in the cap, which can be employed to shape and stabilize an ionized plasma generated within or adjacent to the cap for any therapeutic purpose in humans or animals. Therapeutic treatments specifically contemplated within the scope of the invention include, but are not limited to, tissue de-vitalization, coagulation, carbonization, vaporization or tissue layer surface chemical reactions supported by active ionic and molecular products produced by the cap associated non-thermal ionized plasma (Fridman et al. (2005)). Additionally, the invention includes any device or devices capable of generating an electromagnetic field in the vicinity of the cap constrained or associated ionized plasma through integration of an electromagnetic device or devices into the design of the cap, through attachment of the electromagnetic device or devices to the cap, or through positioning of the electromagnetic device devices in proximity to the cap in such a manner that the generated electromagnetic field can influence the ionized plasma, its' ignition device, or the ignition device supporting mechanism. Finally, the invention includes any modification to the cap edge profile (such as squaring or angling) or to the cap open end shape the purpose of which is to enhance or facilitate debridement of tissue coagulum.

The varying degree of desiccation and thickness of coagulum, which builds up during the plasma coagulation process using APC for Barrett's epithelium, progressively impairs uniformity of tissue conductivity and hence diminishes the precision and uniformity of treatment effect. It was observed that use of a cap of the invention to periodically scrape off coagulum (debridement) during the treatment process greatly improved the uniformity and precision of the treatment effect and permitted ionized plasma ignition and maintenance with the use of extremely low treatment power settings. It was further observed that it was much easier to stabilize the distance between the plasma ignition source (the tip of the probe) and the target tissue during treatment with a cap of the invention fitted onto the endoscope. This innovation, using a cap of the invention on an endoscope, permitted successful ablation of Barrett's epithelium utilizing power settings of about 1 to 20 Watts. These power settings were 40% (or less) of the power settings currently recommended for treatment of Barrett's epithelium by two different APC generator manufacturers and 17% (or less) of the published power settings for high power ablation of Barrett's esophagus or epithelium. It was further observed that relatively large areas of epithelium could be removed with little or no post procedure pain or squealae. Furthermore, the use of very low power settings to ignite and drive the ionized plasma resulted in a translucent plasma which permitted near complete visualization of the target tissue during treatment, a circumstance not possible with any other tissue ablation technology including standard APC methodology, which creates a blindingly bright plasma. The ability to visualize the target tissue during treatment greatly enhances the precision of treatment.

Design modifications with respect to cap length, diameter, and shape provide significant further improvements in the ability to stabilize the distance between the probe tip and the target tissue during treatment. Furthermore, different methods of delivering gas to and venting gas from the cap and positioning of the plasma ignition device further enhance control of the ionized plasma behavior and further minimize the power necessary to ignite and maintain the plasma. For instance, by connecting the gas delivery system and ignition device to the cap, rather than passing these system components through the accessory channel of the endoscope in the form of a probe, the ignition device at the tip of the probe can be positioned ideally with respect to tissue surface and cap walls so as to maximize plasma flow to the target tissue and minimize episodic plasma flow to the cap walls (FIGS. 1A-1B, 2A-2B, 3, and 4A-4C). Additionally, the endoscope accessory channel is free for other uses such as coagulum removal because it no longer need be used as a conduit for the probe. The need to periodically remove the probe from the endoscope accessory channel to physically clear coagulum from its tip during treatment is also eliminated. In addition, cap design to include a probe deflection shelf to better center the probe with respect to the treatment field (FIGS. 8A and 8B) can preserve the current probe-through-the-scope methodology whose major advantage is the real-time ability to alter probe-tissue distance. Uniformity of plasma effect can be provided by routing argon gas into a hollow cap and delivering it to the cap confined treatment space through a multiported system (FIG. 5). Real time adjustments to the shape of a cap confined plasma can be provided through the use of electromagnetic fields generated by devices incorporated into the cap or placed adjacent to it (FIGS. 6A and 6B). Real time electromagnetic plasma shaping provides for an unprecedented level of therapeutic control.

Iterative removal of accumulated coagulum is an integral part of making low power generator settings work for APC tissue layer ablation and is hence an integral and novel aspect of cap design. Current open cap end edge profiles are generally rounded to avoid tissue injury. In one embodiment, open cap end edge profiles are squared or are configured with internal or external tapered lips (FIGS. 9A-9D) to enhance the ability of the caps to successfully remove tissue coagulum (debridement).

As shown in FIGS. 6A and 6B, additional embodiments of the cap can include multiple gas port associated ignition devices arrayed circumferentially around the distal end of the cap which can be fired in rapid sequence.

As shown in FIGS. 8A and 8B, the same basic modifications (gas venting holes and probe deflection shelf) can be utilized with angled and spherical embodiments. The probe deflection shelf is positioned such that when the cap is attached to the endoscope and properly oriented the shelf will position the tip of the probe toward the center of the field as defined by the boundaries of the cap without obstructing the view from the endoscopes objective lens.

The subject invention also concerns an endoscope comprising a cap of the invention.

The subject invention also concerns kits comprising, in one or more containers, an endoscopic cap of the invention. In one embodiment, the cap is provided sterile in a container or package. In one embodiment, the cap is provided as a disposable, one use product. In one embodiment, a kit of the invention includes instructions or packaging materials that describe how to install and/or how to use a cap on an endoscope in a patient. Containers of the kit can be of any suitable material, e.g., glass, plastic, paper, metal, etc., and of any suitable size, shape, or configuration. As noted above, the container and the cap provided therein can be provided in a sterile form.

The subject invention also concerns methods of using an endoscope comprising a cap of the invention. In one embodiment, a method of the invention comprises introducing an endoscope of the invention into the body of a person or animal. The endoscope can then be utilized, for example, for tissue debriding, devitalizing, ablating (polyps, malignant tumors, etc.), coagulating, carbonizing, hemostasis (bleeding ulcers, etc.), and/or vaporizing. In one embodiment, the present invention can be used to treat a premalignant condition (e.g., Barrett's esophagus) or a malignant condition (e.g., esophageal cancer).

The methods of the present invention can be used in the treatment of humans and other animals. The other animals contemplated within the scope of the invention include domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 7,517,347
U.S. Pat. No. 6,210,410
U.S. Pat. No. 6,063,084
U.S. published patent application 2009/0024122
U.S. published patent application 2007/0034211
Fridman, A., Chirokov, A., and Gutsol, A. (2005) “Non-thermal atmospheric pressure discharges” J. Phys. D: Appl. Phys., 38:R1-R24.
Ginsberg, G., Barkun, A., Bosco, J., Burdick, J. S., Isenberg, G., Nakao, N., Petersen, B.,

Silverman, W., Slivka, A., and Kelsey, P. (2002) “The argon plasma coagulator” Gastrointestinal Endoscopy, 55(7):807-810.

1. An apparatus to confine, control and/or shape an ionized plasma and remove tissue coagulum for therapeutic purposes, comprising:
a) flexible or rigid endoscope;
b) a cap secured to said endoscope;
c) a gas delivery system and ignition device connected to said cap;
d) a probe based gas delivery and ignition system brought into the confinement space of the cap.

2 . The apparatus according to claim 1, further comprising:
a) said cap having one or more venting holes.

3. The apparatus according to claim 1, further comprising:
a) said apparatus permitting low power (1to 20 Watts) ignition and maintenance of an ionized plasma.

4. The apparatus according to claim 1, further comprising:
a) said cap having varying cap length, diameter, and shape.

5. The apparatus according to claim 1, further comprising:
a) one or more electromagnetic field creating devices connected to said cap.

6. (canceled)

7. A method to control and shape an ionized plasma and remove tissue coagulum for therapeutic purposes, comprising the steps of:
a) fitting a suitably designed cap onto an endoscope; and
b) scraping off coagulum (debridement) during the treatment process with the cap.

8-9. (canceled)

10. An endoscopic cap, wherein said cap comprises a generally cylindrical cap body having a distal end portion with a distal end opening and having a proximal end portion with a proximal end opening which can operably connect with the end of an endoscope.

11. The cap according to claim 10, wherein the diameter of said distal end opening is larger than the diameter of said proximal end opening.

12. The cap according to claim 11, wherein the wall of said cap body tapers from said proximal end to said distal end wherein said distal portion of said cap body is conical-shaped.

13. The cap according to claim 11, wherein said distal portion of said cap body is U-shaped.

14. The cap according to claim 11, wherein said distal portion of said cap body is round or semi-spherical shaped.

15. The cap according to claim 10, wherein the wall of said distal end portion of said cap body is hollow, wherein said distal end portion comprises an inner wall and an outer wall defining an internal hollow space in said cap body.

16. The cap according to claim 15, wherein said inner wall of said distal end portion of said cap body comprises one or more gas or fluid delivery ports.

17. The cap according to claim 10, wherein the wall of said cap body comprises one or more venting holes in said distal end portion of said cap body.

18. The cap according to claim 10, wherein said cap comprises a conduit on the exterior or interior of said cap body.

19. The cap according to claim 18, wherein said conduit contains fluid, gas and/or ignition device wiring and, optionally, a wash tube for cleaning an ignition device.

20. The cap according to claim 18, wherein said conduit is in direct communication with said internal hollow space in said cap body.

21. The cap according to claim 10, wherein said distal end of said cap comprises a rounded edge, a flat edge, an internal tapered edge, an external tapered edge, or an internal tapered and external tapered edge.

22. The cap according to claim 10, wherein a plasma ignition device for producing an ionized plasma is provided inside of said distal portion of said cap body.

23. The cap according to claim 10, wherein an electromagnetic device is attached or embedded in the inner wall of said distal portion of said cap body.

24. The cap according to claim 10, wherein said cap body comprises a probe deflection shelf to position the tip of a probe toward the center of the field defined by the boundaries of said cap body.

25. (canceled)

26. The cap according to claim 10, wherein said distal end opening is oblique relative to the insertion direction of the endoscope.

27. An endoscope comprising an endoscopic cap, wherein said cap comprises a generally cylindrical cap body having a distal end portion with a distal end opening and having a proximal end portion with a proximal end opening which can operably connect with the end of an endoscope.

28-30. (canceled)

31. A method for treating a person or animal, comprising introducing an endoscope into the body of the person or animal and using said endoscope to perform a therapeutic treatment, wherein said endoscope comprises cap, wherein said cap comprises a generally cylindrical cap body having a distal end portion with a distal end opening and having a proximal end portion with a proximal end opening which can operably connect with the end of an endoscope.

32-35. (canceled)

36. The apparatus according to claim 1, wherein said probe based gas delivery and ignition system does not utilize an accessory channel of said endoscope.
